SEQUENCE LISTING (8 sequences)

SEQ ID NO: 1 - DNA, 244 bp, Herpes simplex virus 1
atgcgccagggcgcccccgcgcgggggtgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccttggcgccgccccaacgggggacccgaaaccgaagaagaacaaaaaaccgaaaaacccaacgccgccac

SEQ ID NO: 2 - DNA, 420 bp, Herpes simplex virus 2
tctgcaggcagacggcggccatgaccccgccgagcagctgccggagcgcggggcaggcgtcgcacgcgtccggcaccaggcgctccagcacggcccgggcccagggctccgagggggcggccgccaccagcgcgtccagcctttccaggcccgcccgcccccgggcttccggcagcccggcctccccgaggcccgcgagggcggccaggagctgggcctggagcccggagaaacaaaaccgcgccgtccagaccggcccgacggccgccggggggtcgagtagttggatggtggtggccgtggggtgccaccgcgcgaccgcttcccgaaaggcgggcaggaggcggccggccgcctccgaggccacggccggccatgcccgcgggggcaggacgaccctggcgcccaccgcgggccagg

SEQ ID NO: 3 - DNA, 18 bp, Artificial Sequence
  Description of Artificial Sequence Synthetic primer
gccggtggttcgtcgtat

SEQ ID NO: 4 - DNA, 22 bp, Artificial Sequence
  Description of Artificial Sequence Synthetic primer
tttttgttcttcttcggtttcg

SEQ ID NO: 5 - DNA, 23 bp, Artificial Sequence
  Description of Artificial Sequence Synthetic probe
ctcttggggttgacgctgggggt

SEQ ID NO: 6 - DNA, 17 bp, Artificial Sequence
  Description of Artificial Sequence Synthetic primer
gcgtccagcctttccag

SEQ ID NO: 7 - DNA, 20 bp, Artificial Sequence
  Description of Artificial Sequence Synthetic primer
gccaccaccatccaactact

SEQ ID NO: 8 - DNA, 20 bp, Artificial Sequence
  Description of Artificial Sequence Synthetic probe
cctggagcccggagaaacaa

(19) United States
(12) Patent Application Publication
(10) Pub. No.: US 2007/0141559 A1
(43) Pub. Date: Jun. 21, 2007

(54) Methods for detecting and typing herpes simplex virus

(75) Inventor: Maurice Exner, Mission Viejo, CA (US)

Correspondence Address: FOLEY & LARDNER LLP, P.O. BOX 80278, SAN DIEGO, CA, 92138-0278, US

(73) Assignee: Quest Diagnostics Inc

(21) Appl. No.: 11/314,202

(22) Filed: Dec. 20, 2005

(51) Int. Cl. C12Q 1/70; C12Q 1/68

(52) U.S. Cl. 435/5; 435/6

(57) ABSTRACT

Detailed herein are methods of detecting herpes simplex virus and differentiating between types 1 and 2 by simultaneous detection of type-specific gene sequences. In particular aspects, individuals infected with HSV-1 can be distinguished from those infected with HSV-2 by amplification and detection of the HSV-1 glycoprotein B gene or the HSV-2 UL-8 gene. Primers and probes for the differential detection of HSV-1 and HSV-2 are provided.

### FIELD OF THE INVENTION

The present invention relates to methods of viral diagnostics and, in particular, detection of herpes simplex virus.

### BACKGROUND

Herpes simplex virus (HSV) is part of the larger herpes virus family, including varicella-zoster virus (VZV), Epstein-Barr virus (EBV) and the cytomegalovirus (CMV). Herpes simplex viruses contain a double-stranded DNA genome and replicate in the cells of infected hosts. All known herpes viruses have three major classes of genes, alpha, beta, and gamma, which have the same order of expression during the viral life cycle. Expression of viral gene products occurs in a distinct order during viral replication. HSV genes are organized within the genome in a specific arrangement with regulatory elements (e.g., promoters) immediately upstream of the open reading frames.

Alpha genes, or immediate-early genes, are the first genes expressed following infection and do not require any other viral product for expression. Beta or early genes, however, are expressed only after alpha genes, the latter gene products being transcriptional activators of beta gene expression. Fourteen genes of the HSV-1 genome have been characterized as beta genes (i.e., UL2, UL5, UL8, UL9, UL12, UL23, UL29, UL30, UL39, UL40, UL42, UL50, UL52, and UL53). Beta gene products are primarily enzymes involved in viral nucleic acid synthesis and metabolism. Gamma or late genes encode structural components of the virus and are generally expressed following viral DNA synthesis.

There are two main types of herpes simplex virus, herpes simplex 1 (HSV-1) and herpes simplex 2 (HSV-2). Both HSV-1 and HSV-2 can cause disease in humans and exposure or infection is fairly common in adult populations. Up to 80% of the U.S. adult population has been exposed to HSV-1 and approximately 20% of the U.S. population has contracted HSV-2 infections. Clinical symptoms can present as fever, headache, malaise, myalgia, and cold sores/lesions that cause pain, itching, dysuria and vaginal or urethral discharge.

Rapid detection of HSV is important for early diagnosis and treatment of infected individuals. Furthermore, distinguishing between infection caused by HSV-1 and HSV-2 assists in prescribing effective antiviral therapy.

Several laboratory techniques are in clinical use for detecting HSV infection. Conventional cell culture methods require 2-3 days for a positive result and up to 2 weeks to confirm a negative result. Furthermore, some clinical samples (e.g., cerebrospinal fluid (CSF)) are not amenable to testing with cell culture methods. Distinguishing HSV-1 from HSV-2 by serology is difficult. Polymerase chain reaction (PCR) is now recognized as the standard for detecting HSV infection, particularly in central nervous system (CNS) infections.

### SUMMARY OF THE INVENTION

Provided herein are methods of identifying and distinguishing HSV-1 from HSV-2 in a sample. This method is accomplished through assaying a nucleic acid-containing sample for two different target gene sequences, one sequence is characteristic of HSV-1 and the other is characteristic of HSV-2. Detection of the first gene is indicative of the presence of HSV-1 nucleic acids, whereas detection of the second gene is indicative of the presence of HSV-2 nucleic acids.

Provided herein are methods of diagnosing an HSV infection and distinguishing between infection by HSV-1 and HSV-2 in a patient. This method is accomplished through assaying a nucleic acid-containing biological sample for two different gene sequences, one sequence is characteristic of HSV-1 and the other is characteristic of HSV-2. Detection of the first gene is indicative of HSV-1 infection, whereas detection of the second gene is indicative of HSV-2 infection.

In particular aspects of either of the above methods, a sample obtained from a patient is assayed for the presence or absence of target sequences from two different genes by amplification and detection of the resulting amplification products. In a preferred embodiment, amplification of target nucleic acids is accomplished by polymerase chain reaction (PCR). Detection of the amplification products is accomplished by hybridization to an oligonucleotide probe, specific for the target sequence. Detection of the amplification product corresponding to the first gene is indicative of the presence of HSV-1 nucleic acids, and thus HSV-1 infection, whereas detection of the amplification product corresponding to the second gene is indicative of the presence of HSV-2 nucleic acids and thus, HSV-2 infection.

In a preferred embodiment, methods are used to detect HSV and to differentiate between HSV-1 and HSV-2 by amplifying nucleic acid molecules encoding HSV-1 glycoprotein B and HSV-2 UL-8.

Amplification of the two genes may be performed simultaneously in a single reaction vessel. In this case, the probes would preferably be distinguishably labeled. Alternatively, the amplification can be in parallel in separate reaction vessels. In such case the probes could have the same or a nondistinguishing label.

Primers may be designed to amplify the target sequences. These primers are preferably “type-specific,” that is, they will hybridize to and amplify the target sequence of one HSV type but not the other. For example, a primer whose 3′ end anneals to a sequence of one HSV type but not the corresponding sequence of the other HSV type, should help to amplify the sequence to which the 3′ end anneals but not to the other sequence. In another example, a primer may match the target sequence from one HSV type exactly but have some mismatches with the corresponding region of the other HSV type. A “mismatch” is defined as a base pairing that does not follow Watson-Crick pairing rules.

Alternatively, primers may be designed that are not type-specific. That is, primers that amplify a sequence from both HSV-1 and HSV-2 may be used. In such a case, the identity of the amplified HSV sequence can be determined by using an oligonucleotide probe designed to hybridize with only one HSV type but not the other.

Thus, for the gB gene, a primer pair can be designed to hybridize to the exemplary segment of the gB gene as shown in FIG. 1 (SEQ ID NO:1). A forward primer can hybridize to SEQ ID NO:1 between nucleotides 1 and 45, more preferably between positions 22 and 39 while a reverse primer can hybridize to SEQ ID NO:1 between positions 70 and 115, more preferably between 97 and 113. One approach is to use a forward primer, SEQ ID NO:3 and a reverse primer, SEQ ID NO:4, to amplify a 193 bp region of the HSV-1 gB gene.

In some embodiments, a primer pair is designed to hybridize to the specified segment of the UL-8 gene as shown in FIG. 2 (SEQ ID NO:2). For example, the forward primer can be designed to hybridize to SEQ ID NO:2 between nucleotides 19200 and 19375, or between positions 19250 and 19350, or between nucleotides 19325 and 19350. A reverse primer can be designed to hybridize to SEQ ID NO:2 between positions 19440 and 19600, or between 19450 and 19550, or between nucleotides 19450 and 19500. One approach is to use a forward primer, SEQ ID NO:6 and a reverse primer, SEQ ID NO:7 to amplify a 167 bp region of the HSV-2 UL-8 gene.

Exemplary oligonucleotides which may be used as primers to amplify a region of the HSV-1 gB gene include SEQ ID NO:3 (5′-GCCGGTGGTTCGTCGTAT-3′) and SEQ ID NO:4 (5′-TTTTTGTTCTTCTTCGGTTTCG-3′).

Exemplary oligonucleotides which may be used as primers to amplify a region of the HSV-2 UL-8 gene include SEQ ID NO:6 (5′-GCGTCCAGCCTTTCCAG-3′) and SEQ ID NO:7 (5′-GCCACCACCATCCAACTACT-3′).

Other exemplary oligonucleotide primers are approximately 15-100 nucleotides in length and comprise SEQ ID NO:3, SEQ ID NO:4, SEQ ID NO:6, and SEQ ID NO:7. Still other exemplary oligonucleotide primers include an oligonucleotide sequence that hybridizes to the complement of a 15-100 nucleotide sequence and that comprises the complement of SEQ ID NO:1 or SEQ ID NO:2. Such oligonucleotide may be substantially purified. Table 1 shows the sequence of primers and a probe for amplifying and detecting a region of the HSV-1 gB gene and a region of the HSV-2 UL-8 gene.

TABLE 1
Primers/probes for amplifying and detecting
regions of the HSV-1 glycoprotein B gene and
the HSV-2 UL-8 gene

| | Probe | |
|---|---|---|
| SEQ ID NO: | Name | Probe Sequence |
| SEQ ID NO:5 | HSV-1 | 5′-CTCTTGGGGTTGACGCTGGGGGT-3′ |
| SEQ ID NO:8 | HSV-2 | 5′-CCTGGAGCCCGGAGAAACAA-3′ |
| | Primer | |
| SEQ ID NO: | Name | Primer Sequence |
| SEQ ID NO:3 | HSV-1 | 5′-GCCGGTGGTTCGTCGTAT-3′ |
| | forward | |
| SEQ ID NO:4 | HSV-1 | 5′-TTTTTGTTCTTCTTCGGTTTCG-3′ |
| | reverse | |
| SEQ ID NO:6 | HSV-2 | 5′-GCGTCCAGCCTTTCCAG-3′ |
| | forward | |
| SEQ ID NO:7 | HSV-2 | 5′-GCCACCACCATCCAACTACT-3′ |
| | reverse | |

Oligonucleotide probes can be designed which are between about 10 and about 100 nucleotides in length and hybridize to the amplified region. Oligonucleotides probes are preferably 12 to 70 nucleotides; more preferably 15-60 nucleotides in length; and most preferably 15-25 nucleotides in length. The probe may be labeled. In one example, SEQ ID NO:5 can be used as an oligonucleotide probe to detect the gB gene of HSV-1 amplified by forward and reverse primers as set forth in SEQ ID NO:3 and SEQ ID NO:4, respectively. In another example, SEQ ID NO:8 can be used as an oligonucleotide probe to detect the UL-8 gene of HSV-2 amplified by forward and reverse primers as set forth in SEQ ID NO:6 and SEQ ID NO:7, respectively.

As used herein, “sample” or “test sample” refers to any liquid or solid material that can assayed for HSV nucleic acids. In preferred embodiments, a test sample is obtained from a biological source (i.e., a “biological sample”), a tissue sample or bodily fluid from an animal, most preferably from a human. Preferred sample tissues include, but are not limited to, cerebrospinal fluid (CSF), swabs of genital lesions, oral lesions, or lesions at other body sites, swabs in transport media, body fluids, body fluids, plasma, serum, amniotic fluid, pericardial fluid, pleural fluid, urine, eye fluid, sputum, or bronchial wash.

As used herein, “nucleic acid” refers broadly to segments of a chromosome, segments or portions of DNA, cDNA, and/or RNA. Nucleic acid may be derived or obtained from an originally isolated nucleic acid containing sample from any source (e.g., isolated from, purified from, amplified from, cloned from, reverse transcribed from sample DNA or RNA).

As used herein, “target nucleic acid” or “target sequence” refers to a sequence to be amplified and/or detected. These include the original nucleic acid sequence to be amplified, its complementary second strand of the original nucleic acid sequence to be amplified, and either strand of a copy of the original sequence which is produced by the amplification reaction. Copies of the target sequence which are generated during the amplification reaction are referred to as amplification products, amplimers or amplicons. Target sequences may be composed of segments of a chromosome, a complete gene with or without intergenic sequence, segments or portions a gene with or without intergenic sequence, or sequence of nucleic acids to which probes or primers are designed. Target nucleic acids may include wild type sequences, nucleic acid sequences containing mutations, deletions or duplications, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA. As used herein target nucleic acid may be native DNA or a PCR amplified product. Target sequence includes sequences in HSV nucleic acids.

As used herein, “genomic nucleic acid” or “genomic DNA” refers to some or all of the DNA from the nucleus of a cell. Genomic DNA may be intact or fragmented (e.g., digested with restriction endonucleases by methods known in the art). Genomic DNA may include sequence from all or a portion of a single gene or from multiple genes, sequence from one or more chromosomes, or sequence from all chromosomes of a cell. As is well known, genomic nucleic acid includes gene coding regions, introns, 5′ and 3′ untranslated regions, 5′ and 3′ flanking DNA and structural segments such as telomeric and centromeric DNA, replication origins, and intergenic DNA. Genomic nucleic acid may be obtained from the nucleus of a cell, or recombinantly produced. Genomic DNA also may be transcribed from DNA or RNA isolated directly from a cell nucleus. PCR amplification also may be used. Methods of purifying DNA and/or RNA from a variety of samples are well-known in the art.

As used herein, “viral genomic nucleic acid” refers to the genetic material of a virus such as HSV. Viral genomic nucleic acids can be RNA or DNA and can be obtained, for example, from an infected host cell, recombinantly produced or by PCR amplification.

As used herein, “oligonucleotide” refers to a short polymer composed of deoxyribonucleotides, ribonucleotides or any combination thereof. Oligonucleotides useful in the methods described herein are generally between about 10 and about 100 nucleotides in length. Oligonucleotides are preferably 15 to 70 nucleotides long, with 20 to 26 nucleotides being the most common. The single letter code for nucleotides is as described in the U.S. Patent Office Manual of Patent Examining Procedure, section 2422, table 1. In this regard, the nucleotide designation “R” means guanine or adenine, “Y” means thymine (uracil if RNA) or cytosine; and “M” means adenine or cytosine. An oligonucleotide may be used as a primer or as a probe.

As used herein, “substantially purified” in reference to an oligonucleotide sequence does not require absolute purity. Instead, it represents an indication that the oligonucleotide sequence is relatively more pure than in its natural environment or the environment in which it was initially produced. A “substantially purified” oligonucleotide is preferably greater than 50% pure, more preferably at least 75% pure, and most preferably at least 95% pure. Oligonucleotides may be obtained by a number of methods including, for example, laboratory synthesis, restriction enzyme digestion or PCR.

As used herein, nucleic acid sequences that have “high sequence identity” have identical nucleotides at least at about 50% of aligned nucleotide positions, preferably at least at about 75% of aligned nucleotide positions, more preferably at least at about 90% of aligned nucleotide positions, and most preferably at least at about 95% of aligned nucleotide positions.

As used herein, an oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization/washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity. An oligonucleotide which is “gene-specific” is specific, as defined above, for the target gene to which it has been designed and will not substantially hybridize to other genes.

As used herein, “hybridize” or “specifically hybridize” refers to a process where two complementary nucleic acid strands anneal to each in accordance with Watson-Crick base pairing rules. Hybridizations are typically and preferably conducted with probe-length nucleic acid molecules, preferably 20-100 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to determine the appropriate stringency of hybridization/washing conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

As used herein, “complement” means the complementary sequence to a nucleic acid according to standard Watson/Crick pairing rules. A complement sequence can also be a sequence of RNA complementary to the DNA sequence or its complement sequence, and can also be a cDNA.

As used herein, “substantially complementary” refers to sequences that are fully complementary or that are partially complementary such that the two sequences will hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

As used herein, “coding sequence” means a sequence of a nucleic acid which codes for a protein. Coding sequence may refer to the DNA sequence found in genomic DNA or cDNA or may refer to mRNA. Coding sequences include exons in a genomic DNA or immature primary RNA transcripts, which are joined together by the cell's biochemical machinery to provide a mature mRNA, or fragments of any of these sequences. The anti-sense strand is the complement of such a nucleic acid, and the encoding sequence can be deduced therefrom.

As used herein, “non-coding sequence” means a sequence of a nucleic acid or its complement, or a part thereof, that is not transcribed into protein in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

As used herein, “amplification” or “amplify” as used herein means one or more methods known in the art for copying a target nucleic acid, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Inis et al., Eds., Academic Press, San Diego, Calif. 1990, pp 13-20; Wharam et al., Nucleic Acids Res. Jun. 1, 2000;29(11):E54-E54; Hafner et al., Biotechniques April 2001;30(4):852-6, 858, 860 passim; Zhong et al., Biotechniques April 2001;30(4):852-6, 858, 860 passim.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal amplification.

As used herein, “sense strand” means the strand of double-stranded DNA (dsDNA) that includes at least a portion of a coding sequence of a functional protein. “Anti-sense strand” means the strand of dsDNA that is the reverse complement of the sense strand.

As used herein, a “forward primer” is a primer that anneals to the anti-sense strand of dsDNA. A “reverse primer” anneals to the sense-strand of dsDNA.

As used herein, sequences that have “high sequence identity” have identical nucleotides at least at about 50% of aligned nucleotide positions, preferably at least at about 75% of aligned nucleotide positions, more preferably at least at about 90% of aligned nucleotide positions, and most preferably at least at about 95% of aligned nucleotide positions.

As used herein “TaqMan PCR detection system” refers to a method for real time PCR. In this method, a TaqMan probe which hybridizes to the nucleic acid segment amplified is included in the PCR reaction mix. The TaqMan probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polyrnerase cleaves the probe thereby allowing the donor fluorophore to emit fluorescence which can be detected.

### BRIEF DESCRIPTION OF THE FIGURES

FIG. 1. Exemplary sequence (SEQ ID NO: 1) of a region of HSV-1 glycoprotein B cDNA (corresponding to bases 1-243 of GenBank ID g6165611) showing the preferred locations for hybridizing PCR primers (shaded regions), and a preferred location for a hybridizing probe (bold underlined).

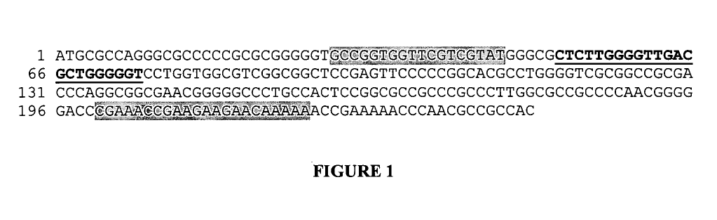

FIG. 2. Exemplary sequence (SEQ ID NO:2) of a region of the HSV-2-UL-8 gene (numbering refers to numbering of HSV-2 genome) showing the preferred locations for hybridizing PCR primers (shaded regions), and a preferred location for a hybridizing probe (bold underlined).

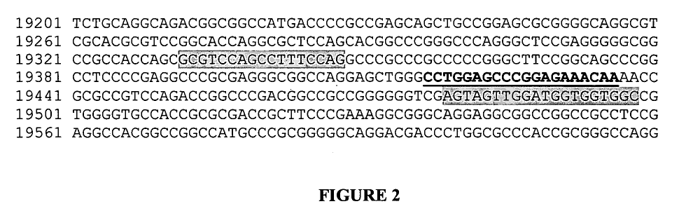

### DETAILED DESCRIPTION OF THE INVENTION

In accordance with the present invention, there are provided methods of detecting and distinguishing between HSV-1 and HSV-2 in a sample and using such methods for diagnosis of infection in an individual. Thus, described are methods of detecting type-specific regions of two different genes from the genomes of HSV-1 and HSV-2.

### Sample Preparation

The methods of the present invention can be used to diagnose an infection by HSV-1 or HSV-2 in an individual by detecting the presence of HSV-1 or HSV-2 nucleic acids in a test sample. Therefore, the method may be performed using any biological sample containing HSV viral nucleic acids. Examples of biological samples include tissue samples or any bodily fluid containing cells infected with HSV. Biological samples may be obtained by standard procedures and may be used immediately or stored, under conditions appropriate for the type of biological sample, for later use.

Methods of obtaining test samples are well known to those of skill in the art and include, but are not limited to, aspirations, drawing of blood or other fluids, swabbing of tissues, and the like. The test sample may be obtained from individual or patient. The test sample may contain cells or fluid obtained from a patient suspected being infected with herpes simplex virus. The test sample may be a cell-containing liquid or a tissue. Samples may include, but are not limited to, sputum, bronchial wash, whole blood, swabs, ocular swab, genital specimen, dermal specimen, pap smear, body fluids, cerebrospinal fluid (CSF), serum, plasma, amniotic fluid, pericardial fluid, pleural fluid, urine, and eye fluid. Samples may also be processed, such as fractionation, purification, or cellular organelle separation.

In particular embodiments, viral DNA or RNA, including messenger RNA (mRNA) can be used as a template for amplification. Template nucleic acid need not be purified and may comprise only a minor fraction of a complex mixture, (e.g., HSV nucleic acids contained in human host cells). DNA or RNA may be extracted from a biological sample such as ocular swabs, genital specimens, dermal specimens, pap smears, amniotic fluid, and CSF by routine techniques such as those described in Diagnostic Molecular Microbiology: Principles and Applications (Persing et al. (eds), 1993, American Society for Microbiology, Washington D.C.). Template nucleic acids can be obtained from any number of sources, such as plasmids, or natural sources including bacteria, yeast, viruses, organelles, or higher organisms such as infected human or animal cells.

Viscous samples such as sputum and bronchial wash may be liquefied prior to extraction of DNA by adding N-acetyl-1-cysteine (NALC) suspended in a solution of citrate and NaOH. Once the sample is liquefied, cells can be collected subjected to a standard DNA extraction procedure using known methods or any of a number of commercially available DNA extraction kits (e.g., MagNA Pure LC DNA Isolation Kit).

### Amplification of Nucleic Acids

Viral nucleic acids containing target sequences may be amplified by various methods known to the skilled artisan. Amplification methods suitable for use with the present methods include, for example, polymerase chain reaction (PCR), ligase chain reaction (LCR), transcription-based amplification system (TAS), nucleic acid sequence based amplification (NASBA) reaction, self-sustained sequence replication (3SR), strand displacement amplification (SDA) reaction, boomerang DNA amplification (BDA), Q-beta replication, or isothermal nucleic acid sequence based amplification. These methods of amplification each described briefly below and are well-known in the art.

PCR is a technique for making many copies of a specific template DNA sequence. The reaction consists of multiple amplification cycles and is initiated using a pair of primer oligonucleotides that hybridize to the 5′ and 3′ ends of the sequence to be copied. The amplification cycle includes an initial denaturation, and up to 50 cycles of annealing, strand elongation (or extension) and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan, et al, Journal of Clinical Microbiology, 33(3):556-561(1995). Briefly, a PCR reaction mixture includes two specific primers, dNTPs, approximately 0.25 U of Taq polymerase, and 1× PCR Buffer. For every 25 μl PCR reaction, 2 μl sample (e.g., isolated DNA from target organism) is added and amplified using a thermal cycler.

LCR is a method of DNA amplification similar to PCR, except that it uses four primers instead of two and uses the enzyme ligase to ligate or join two segments of DNA. LCR can be performed as according to Moore et al., Journal of Clinical Microbiology 36(4 :1028-1031 (1998). Briefly, an LCR reaction mixture contains two pair of primers, dNTP, DNA ligase and DNA polymerase representing about 90 μl, to which is added 100 μl of isolated nucleic acid from the target organism. Amplification is performed in a thermal cycler (e.g., LCx of Abbott Labs, North Chicago, Ill.).

TAS is a system of nucleic acid amplification in which each cycle is comprised of a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS 86:1173-7 (1989). Briefly, extracted RNA is combined with TAS amplification buffer and bovine serum albumin, dNTPs, NTPs, and two oligonucleotide primers, one of which contains a PBS. The sample is heated to denature the RNA template and cooled to the primer annealing temperature. Reverse transcriptase (RT) is added the sample incubated at the appropriate temperature to allow cDNA elongation. Subsequently T7 RNA polymerase is added and the sample is incubated at 37° C. for approximately 25 minutes for the synthesis of RNA. The above steps are then repeated. Alternatively, after the initial cDNA synthesis, both RT and RNA polymerase are added following a 1 minute 100° C. denaturation followed by an RNA elongation of approximately 30 minutes at 37° C. TAS can be also be performed on solid phase as according to Wylie et al., Journal of Clinical Microbiology, 36(12):3488-3491 (1998). In this method, nucleic acid targets are captured with magnetic beads containing specific capture primers. The beads with captured targets are washed and pelleted before adding amplification reagents which contains amplification primers, dNTP, NTP, 2500 U of reverse transcriptase and 2500 U of T7 RNA polymerase. A 100 μl TMA reaction mixture is placed in a tube, 200 μl oil reagent is added and amplification is accomplished by incubation at 42° C. in a waterbath for one hour.

NASBA is a transcription-based amplification method which amplifies RNA from either an RNA or DNA target. NASBA is a method used for the continuous amplification of nucleic acids in a single mixture at one temperature. For example, for RNA amplification, avian myeloblastosis virus (AMV) reverse transcriptase, RNase H and T7 RNA polymerase are used. This method can be performed as according to Heim, et al., Nucleic Acids Res., 26(9 :2250-2251 (1998). Briefly, an NASBA reaction mixture contains two specific primers, dNTP,.NTP, 6.4 U of AMV reverse transcriptase, 0.08 U of Escherichia coli Rnase H, and 32 U of T7 RNA polymerase. The amplification is carried out for 120 min at 41° C. in a total volume of 20 μl.

In a related method, self-sustained sequence-replication (3SR) reaction, isothermal amplification of target DNA or RNA sequences in vitro using three enzymatic. activities: reverse transcriptase, DNA-dependent RNA polymerase and Escherichia coli ribonuclease H. This method may be modified from a 3-enzyme system to a 2-enzyme system by using human immunodeficiency virus (HIV)-1 reverse transcriptase instead of avian myeloblastosis virus (AMV) reverse transcriptase to allow amplification with T7 RNA polymerase but without E. coli ribonuclease H. In the 2-enzyme 3SR, the amplified RNA is obtained in a purer form compared with the 3-enzyme 3SR (Gebinoga & Oehlenschlager European Journal of Biochemistry, 235:256-261, 1996).

SDA is an isothermal nucleic acid amplification method. A primer containing a restriction site is annealed to the template. Amplification primers are then annealed to 5′ adjacent sequences (forming a nick) and amplification is started at a fixed temperature. Newly synthesized DNA strands are nicked by a restriction enzyme and the polymerase amplification begins again, displacing the newly synthesized strands. SDA can be performed as according to Walker, et al., PNAS, 89:392-6 (1992). Briefly, an SDA reaction mixture contains four SDA primers, dGTP, dCTP, TTP, dATP, 150 U of Hinc II, and 5 U of exonuclease-deficient of the large fragment of E. coli DNA polymerase O (exo⁻ Klenow polymerase). The sample mixture is heated 95° C. for 4 minutes to denature target DNA prior to addition of the enzymes. After addition of the two enzymes, amplification is carried out for 120 min. at 37° C. in a total volume of 50 μl. Then, the reaction is terminated by heating for 2 minutes at 95° C.

Boomerang DNA amplification (BDA) is a method in which the polymerase begins extension from a single primer-binding site and then makes a loop around to the other strand, eventually returning to the original priming site on the DNA. BDA is differs from PCR through its use of a single primer. This method involves an endonuclease digestion of a sample DNA, producing discrete DNA fragments with sticky ends, ligating the fragments to “adapter” polynucleotides (comprised of a ligatable end and first and second self-complementary sequences separated by a spacer sequence) thereby forming ligated duplexes. The ligated duplexes are denatured to form templates to which an oligonucleotide primer anneals at a specific sequence within the target or marker sequence of interest. The primer is extended with a DNA polymerase to form duplex products followed by denaturation of the duplex products. Subsequent multiple cycles of annealing, extending, and denaturing are performed to achieve the desired degree of amplification (U.S. Pat. No. 5,470,724).

The Q-beta replication system uses RNA as a template. Q-beta replicase synthesizes the single-stranded RNA genome of the coliphage Qβ. Cleaving the RNA and ligating in a nucleic acid of interest allows the replication of that sequence when the RNA is replicated by Q-beta replicase (Kramer & Lizardi Trends Biotechnol. 1991 9(2):53-8, 1991).

A variety of amplification enzymes are well known in the art and include, for example, DNA polymerase, RNA polymerase, reverse transcriptase, Q-beta replicase, thermostable DNA and RNA polymerases. Because these and other amplification reactions are catalyzed by enzymes, in a single step assay that the nucleic acid releasing reagents and the detection reagents should not be potential inhibitors of amplification enzymes if the ultimate detection is to be amplification based.

Preferably, PCR is used to amplify a target sequence of a gene. In this method, two or more oligonucleotide primers that anneal to opposite strands of a target sequence are repetitively annealed to their complementary sequences, extended by a DNA polymerase (e.g., AmpliTaq Gold polymerase), and heat denatured, resulting in exponential amplification of the target nucleic acid sequences. Cycling parameters can be varied, depending on, for example, the melting temperature of the primer or the length of nucleic acids to be extended. The skilled artisan is capable of designing and preparing primers that are appropriate for amplifying a target sequence. The length of the amplification primers for use in the present invention depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill and include considerations described herein. For example, the length of a short nucleic acid or oligonucleotide can relate to its hybridization specificity or selectivity.

In preferred embodiments, a forward primer, SEQ ID NO:3, and a reverse primer, SEQ ID NO:4, are used to amplify a 193 bp region of the HSV-1 glycoprotein B gene. A second forward primer, SEQ ID NO:6, and a second reverse primer, SEQ ID NO:7, are used to amplify a 167 bp region of the HSV-2 UL-8 gene.

Assay controls may be used in the assay for detecting HSV nucleic acid. Positive controls with high concentrations (e.g. 100,000 copies/mL) and low concentrations (1,000 organisms/mL) of viral nucleic acid may be used. A negative control using no template or a template with a sequence unrelated to the target sequences (and therefore unlikely to be amplified by the assay primers) may be used. An internal positive amplification control (IPC) can be included in the sample and may be introduced as part of a primer/probe mastermix to distinguish true target negatives from PCR inhibition.

### Detection of Amplified DNA

Amplification of nucleic acids can be detected by any of a number of methods well-known in the art such as gel electrophoresis, column chromatography, hybridization with a probe, or sequencing.

In a particular aspect, a target sequence from each of two genes is amplified in the same reaction vessel. In this case, the identity of the amplicon(s) can be determined by the size of the amplicon. The separation of amplicons of different sizes can be accomplished by, for example, gel electrophoresis, column chromatography, or capillary electrophoresis. These and other separation methods are well-known in the art. In one example, amplicons of about 10 to about 150 base pairs whose sizes differ by 10 or more base pairs can be separated on a 4% to 5% agarose gel, (a 2% to 3% agarose gel for about 150 to about 300 base pair amplicons)or a 6% to 10% polyacrylamide gel. The separated nucleic acids may be stained with a dye such as ethidium bromide for ease of detection. The size of a particular band or bands in a gel can be determined by comparing to a standard DNA ladder.

In another aspect, a target sequence from each of two genes is amplified in separate reaction vessels. If the amplification is specific, that is, one target sequence is amplified from one HSV type but not the other, detection of amplification alone can be sufficient to distinguish between the two types.

In some embodiments, amplified nucleic acids are detected by hybridization with a gene-specific probe. Probe oligonucleotides, complementary to the amplified target sequence may be used to detect amplified fragments. Amplified nucleic acids for each of the target sequences may be detected simultaneously (i.e., in the same reaction vessel) or individually (i.e., in separate reaction vessels). In preferred embodiments, the amplified DNA is detected simultaneously, using two distinguishably-labeled, gene-specific oligonucleotide probes, one which hybridizes to the first target sequence and one which hybridizes to the second target sequence.

The probe may be detectably labeled by methods known in the art. Useful labels include, e.g., fluorescent dyes (e.g., Cy5®, Cy3®, FITC, rhodamine, lanthamide phosphors, Texas red), ³²P, ³⁵S, ³H, ¹⁴C, ¹²⁵I, ¹³¹I, electron-dense reagents (e.g., gold), enzymes, e.g., as commonly used in an ELISA (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can be directly incorporated into the nucleic acid to be detected, or it can be attached to a probe (e.g., an oligonucleotide) or antibody that hybridizes or binds to the nucleic acid to be detected.

In preferred embodiment the detectable label is a fluorophore. The term “fluorophore” as used herein refers to a molecule that absorbs light at a particular wavelength (excitation frequency), and subsequently emits light of a different, typically longer, wavelength (emission frequency) in response. Suitable fluorescent moieties include the following fluorophores known in the art:
4-acetamido-4′-isothiocyanatostilbene-2,2′disulfonic acid
acridine and derivatives:
   acridine
   acridine isothiocyanate
Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes)
5-(2′-aminoethyl)aminonaphthalene-1-sulfonic acid (EDANS)
4-amino-N-[3-vinylsulfonyl)phenyl]naphthalimide-3,5 disulfonate (Lucifer Yellow VS)
N-(4-anilino-1-naphthyl)maleimide
anthranilamide
Black Hole Quencher™ (BHQ™) dyes (biosearch Technologies)
BODIPY® R-6G, BOPIPY® 530/550, BODIPY® FL
Brilliant Yellow
coumarin and derivatives:
   coumarin
   7-amino-4-methylcoumarin (AMC, Coumarin 120)
7-amino-4-trifluoromethylcouluarin (Coumarin 151)
Cy2®, Cy3®, Cy3.5®, Cy5®, Cy5.5®
cyanosine
4′,6-diaminidino-2-phenylindole (DAPI)
5′,5″-dibromopyrogallol-sulfonephthalein (Bromopyrogallol Red)
7-diethylamino-3-(4′-isothiocyanatophenyl)-4-methylcoumarin
diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansyl chloride)
4-(4′-dimethylaminophenylazo)benzoic acid (DABCYL)
4-dimethylaminophenylazophenyl-4′-isothiocyanate (DABITC)
Eclipse™ (Epoch Biosciences Inc.)
eosin and derivatives:
   eosin
   eosin isothiocyanate
erythrosin and derivatives:
   erythrosin B
   erythrosin isothiocyanate
ethidium
fluorescein and derivatives:
   5-carboxyfluorescein (FAM)
   5-(4,6-dichlorotriazin-2-yl)aminofluorescein (DTAF)
   2′,7′-dimethoxy-4′5′-dichloro-6-carboxyfluorescein (JOE)
   fluorescein
   fluorescein isothiocyanate (FITC)
   hexachloro-6-carboxyfluorescein (HEX)
   QFITC (XRITC)
   tetrachlorofluorescein (TET)
fluorescamine
IR144
IR1446
Malachite Green isothiocyanate
4-methylumbelliferone
ortho cresolphthalein
nitrotyrosine
pararosaniline
Phenol Red
B-phycoerythrin, R-phycoerythrin
o-phthaldialdehyde
Oregon Green®
propidium iodide
pyrene and derivatives:
   pyrene
   pyrene butyrate
   succinimidyl 1-pyrene butyrate
QSY®7, QSY®9, QSY®21, QSY®35 (Molecular Probes)
Reactive Red 4 (Cibacron® Brilliant Red 3B-A)
rhodamine and derivatives:
   6-carboxy-X-rhodamine (ROX)
   6-carboxyrhodamine (R6G)
   lissamine rhodamine B sulfonyl chloride
   rhodamine (Rhod)
   rhodamine B
   rhodamine 123
   rhodamine green
   rhodamine X isothiocyanate
   sulforhodamine B
   sulforhodamine 101

1sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
terbium chelate derivatives

Other fluorescent nucleotide analogs can be used, see, e.g., Jameson, Meth. Enzymol. 278:363-390, 1997; Zhu, Nucl. Acids Res. 22:3418-3422, 1994. U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

The detectable label can be incorporated into, associated with or conjugated to a nucleic acid. Label can be attached by spacer arms of various lengths to reduce potential steric hindrance or impact on other useful or desired properties. See, e.g., Mansfield, Mol. Cell. Probes 9:145-156, 1995.

Detectable labels can be incorporated into nucleic acids by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3® or Cy5® and then incorporated into genomic nucleic acids during nucleic acid synthesis or amplification. Nucleic acids can thereby be labeled when synthesized using Cy3®- or Cy5®-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu, Nat. Biotechnol. 18:345-348, 2000.

Nucleic acid probes may be labeled by non-covalent means known in the art. For example, Kreatech Biotechnology's Universal Linkage System® (ULS®) provides a non-enzymatic labeling technology, wherein a platinum group forms a co-ordinative bond with DNA, RNA or nucleotides by binding to the N7 position of guanosine. This technology may also be used to label proteins by binding to nitrogen and sulfur containing side chains of amino acids. See, e.g., U.S. Pat. Nos. 5,580,990; 5,714,327; and 5,985,566; and European Patent No. 0539466.

The binding of a probe to the marker sequence flanking the tandem repeat region may be determined by hybridization as is well known in the art. Hybridization may be detected in real time or in non-real time.

One general method for real time PCR uses fluorescent probes such as the TaqMan® probes, molecular beacons and scorpions. The probes employed in TaqMan® and molecular beacon technologies are based on the principle of fluorescence quenching and involve a donor fluorophore and a quenching moiety.

The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e. collisional) quenching or by Forster or fluorescence resonance energy transfer (“FRET”). Quenching by FRET is generally used in TaqMan® probes while proximal quenching is used in molecular beacon and scorpion type probes.

In proximal quenching (a.k.a. “contact” or “collisional” quenching), the donor is in close proximity to the quencher moiety such that energy of the donor is transferred to the quencher, which dissipates the energy as heat as opposed to a fluorescence emission. In FRET quenching, the donor fluorophore transfers its energy to a quencher which releases the energy as fluorescence at a higher wavelength. Proximal quenching requires very close positioning of the donor and quencher moiety, while FRET quenching, also distance related, occurs over a greater distance (generally 1-10 nm, the energy transfer depending on R⁻⁶, where R is the distance between the donor and the acceptor). Thus, when FRET quenching is involved, the quenching moiety is an acceptor fluorophore that has an excitation frequency spectrum that overlaps with the donor emission frequency spectrum. When quenching by FRET is employed, the assay may detect an increase in donor fluorophore fluorescence resulting from increased distance between the donor and the quencher (acceptor fluorophore) or a decrease in acceptor fluorophore emission resulting from increased distance between the donor and the quencher (acceptor fluorophore).

TaqMan® probes (Heid et al., 1996) use the fluorogenic 5′ exonuclease activity of Taq polymerase to measure the amount of target or marker sequences in DNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo)benzoic acid (DABCYL). See Tyagi et al., Nature Biotechnology 16:49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase replicates a template on which a TaqMan® probe is bound, its 5′ exonuclease activity cleaves the probe. This ends the activity of quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

TaqMan® assay uses universal thermal cycling parameters and PCR reaction conditions. Because the cleavage occurs only if the probe hybridizes to the target, the fluorescence detected originates from specific amplification. The process of hybridization and cleavage does not interfere with the exponential accumulation of the product. One specific requirement for fluorogenic probes is that there be no G at the 5′ end. A ‘G’ adjacent to the reporter dye quenches reporter fluorescence even after cleavage.

Other methods of probe hybridization detected in real time can be used for detecting amplification a target or marker sequence flanking a tandem repeat region. For example, the commercially available MGB Eclipse™ probes (Epoch Biosciences), which do not rely on a probe degradation can be used. MGB Eclipse™ probes work by a hybridization-triggered fluorescence mechanism. MGB Eclipse™ probes have the Eclipse™ Dark Quencher and the MGB positioned at the 5′-end of the probe. The fluorophore is located on the 3′-end of the probe. When the probe is in solution and not hybridized, the three dimensional conformation brings the quencher into close proximity of the fluorophore, and the fluorescence is quenched. However, when the probe anneals to a target or marker sequence, the probe is unfolded, the quencher is moved from the fluorophore, and the resultant fluorescence can be detected.

Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like. Suitable quenchers include tetra-methylcarboxyrhodamine (TAMRA) 4-(4-dimethylaminophenylazo)benzoic acid (“DABCYL” or a DABCYL analog) and the like. Tetramethylrhodamine (TMR) or 5-carboxyrhodamine 6G (RHD) may be combined as donor fluorophores with DABCYL as quencher. Multiplex TaqMan assays can be performed using multiple detectable labels each comprising a different donor and quencher combination. Probes for detecting amplified sequence in real time may be stored frozen (−10° to −30° C.) as 100 μM stocks. TaqMan probes are available from Applied BioSystems (4316032).

In a preferred embodiment, real time PCR is performed using TaqMan® probes in combination with a suitable amplification/analyzer such as Applied Biosystems (ABI) Prism 7900HT Sequence Detection System. The ABI PRISM® 7900HT Sequence Detection System is a high-throughput real-time PCR system that detects and quantitates nucleic acid sequences. Briefly, TaqMan™ probes specific for the amplified target sequence are included in the PCR amplification reaction. These probes contain a reporter dye at the 5′ end and a quencher dye at the 3′ end. Probes hybridizing to different target sequences are conjugated with a different fluorescent reporter dye. In this way, more than one target sequence can be assayed for in the same reaction vessel. During PCR, the fluorescently labeled probes bind specifically to their respective target sequences; the 5′ nuclease activity of Taq polymerase cleaves the reporter dye from the probe and a fluorescent signal is generated. The increase in fluorescence signal is detected only if the target sequence is complementary to the probe and is amplified during PCR. A mismatch between probe and target greatly reduces the efficiency of probe hybridization and cleavage. The ABI Prism 7700HT or 7900HT Sequence detection System measures the increase in fluorescence during PCR thermal cycling, providing “real time” detection of PCR product accumulation.

Real Time detection on the ABI Prism 7900HT or 7900HT Sequence Detector monitors fluorescence and calculates Rn during each PCR cycle. The threshold cycle, or Ct value, is the cycle at which fluorescence intersects the threshold value. The threshold value is determined by the sequence detection system software or manually.

Amplified nucleic acids can be detected by hybridization methods such as Southern blotting. In this case, amplified nucleic acids are subjected to gel electrophoresis, transferred to cellulose or nylon membrane and detected by hybridization with a labeled probe.

### Correlation of Detection of a Target Sequence with Diagnosis

The detection of amplified target sequences characteristic of HSV-1 in a biological sample from an individual, is indicative of infection by HSV-1. Detection of amplified target sequences characteristic of HSV-2 in a biological sample from an individual, is indicative of infection by HSV-2. Detection of both targets in the same sample is indicative of infection by both HSV-1 and HSV-2.

The examples below illustrate a standard protocol for performing PCR and analyzing in real time. The TaqMan system of primer labeling is a preferred method of real time detection of PCR amplicons. The following examples serve to illustrate the present invention. These examples are in no way intended to limit the scope of the invention.

### EXAMPLE 1

### Primer/Probe Mastermix Preparation:

A stock solution of primer/probe mastermix was prepared by mixing each of the stock solutions as shown in Table 2.

TABLE 2
Primer/Probe Mastermix.

| | Final |
|---|---|
| | concentration per |
| Component | reaction |
| Sterile Nuclease Free Water | 3.45 mM |
| MgCl₂ (25 mM) | |
| l0x Exo IPC* Mix | 1 x |
| 50x Exo IPC DNA | 1 x |
| HSV-1 Forward Primer (100 μM) | 500 nM |
| HSV-1 Reverse Primer (100 μM) | 500 nM |
| HSV-1 Oligonucleotide Probe | 100 nM |
| (100 μM) | |
| HSV-2 Forward Primer (100 μM) | 500 nM |
| HSV-2 Reverse Primer (100 μM) | 500 nM |
| HSV-2 Oligonucleotide Probe | 150 nM |
| (100 μM) | |
| Total | |

*Exo IPC: Exogenous internal positive control

The primer/probe mastermix stock solution was dispensed into 290 μl aliquots. Each aliquot is combined with 500 μl of Applied Biosystems Universal mastermix and 10 μl of AmpliTaq Gold and is sufficient for 18 reactions. This solution can be stored at −20° C. for 1 year from the date of preparation.

### EXAMPLE 2

### Preparation and DNA Extraction of Clinical Samples.

Swabs of lesions in transport media, CSF, or serum were extracted using the Roche MagNA Pure LC automated nucleic acid extraction system. The samples (0.2 ml) were loaded directly onto the instrument, and purified nucleic acid was extracted using the MagNA Pure LC Total Nucleic Acid Isolation kit.

### EXAMPLE 3

### DNA Amplification

To prepare the final mastermix, 0.5 mL of 2× Mastermix (Applied Biosystems #4304437), and 10 μl AmpliTaq Gold was added to a single vial (290 μl) of stock primer/probe mastermix. The resulting solution was quickly spun for 5 seconds. 40 μl was dispensed into each well of a 96-well plate to be used for PCR. The extracts from the control or biological samples were added to individual wells (10 μl/well) containing the final mastermix and pipetted up and down 2′ to mix the sample and mastermix. The plate was sealed with an optical adhesive cover and transferred to the Applied Biosystems 7700 (or 7900HT) sequence detector.

The thermocycler conditions were as follows:
Stage 1: Hold at 50.0° C. for 2 min.
Stage 2: Hold at 95° C. for 10 min.
Stage 3: Cycle from 95.0° C. for 15 s to 60° C. for 1 min, 50 cycles.
Sample volume: 50 μl.

### EXAMPLE 4

### Data Analysis

The assay as described has been used to detect HSV-1 and HSV-2 nucleic acids in a variety of clinical specimens, including cerebrospinal fluid, swabs in M4 medium, serum, and other body fluids. The assay results were reproducible over the course of multiple runs. Method comparison studies performed to detect HSV-1 and HSV-2 in patient specimens that tested positive by LightCycler PCR assay were performed. This included a comparison with the LightCycler PCR assay. The results support the conclusion that the real-time PCR format described herein is both sensitive and specific, detecting specimens that were shown previously to be positive for HSV.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing“, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

That which is claimed is:

1. A method for detecting and differentiating between HSV-1 and HSV-2 in a sample, said method comprising, detecting a first target sequence specific for HSV-1 when HSV-1 nucleic acids are present in said sample, and detecting a second target sequence specific for HSV-2 when HSV-2 nucleic acids are present in said sample, wherein said first target sequence and said second target sequence are from different genes, and wherein detection of said first target sequence is indicative of an HSV-1 infection and detection of said second target sequence is indicative of an HSV-2 infection.

2. A method according to claim 1, wherein said first target sequence is from the HSV-1 glycoprotein B gene.

3. A method according to claim 1, wherein said second target sequence is from the HSV-2 UL-8 gene.

4. A method according to claim 1, wherein said first target sequence is from the HSV-1 glycoprotein B gene and said second target sequence is from the HSV-2 UL-8 gene.

5. A method according to claim 1, wherein said detecting further comprises, amplifying said first and second target sequences.

6. A method according to claim 5, wherein said amplifying is accomplished with the polymerase chain reaction.

7. A method according to claim 1, wherein said detecting of said first and second target sequences occurs in the same reaction vessel.

8. A method according to claim 1, wherein said detecting of said first and second target sequences occurs in separate reaction vessels.

9. A method according to claim 1, wherein said detecting further comprises, amplifying a region of the HSV-1 glycoprotein B (gB) gene with a first pair of type-specific primers when HSV-1 nucleic acids are present in said sample, amplifying a region of the HSV-2 UL-8 gene with a second pair of type-specific primers when HSV-2 nucleic acids are present in said sample, and
detecting said amplified regions with a first gene-specific probe and a second gene-specific probe.

10. A method according to claim 9, wherein said probes are labeled.

11. A method according to claim 10, wherein said probes are distinguishably labeled.

12. A method according to claim 9, wherein said first pair of type-specific primers comprises a forward primer comprising the sequence set forth in SEQ ID NO:3 and a reverse primer.

13. A method according to claim 9, wherein said first pair of type-specific primers comprises a forward primer and a reverse primer comprising the sequence set forth in SEQ ID NO:4.

14. A method according to claim 9, wherein said second pair of type-specific primers comprises a forward primer comprising the sequence set forth in SEQ ID NO:6 and a reverse primer.

15. A method according to claim 9, wherein said second pair of type-specific primers comprises a forward primer and a reverse primer comprising the sequence set forth in SEQ ID NO:7.

16. A method according to claim 9, wherein said first gene-specific probe comprises the sequence set forth in SEQ ID NO:5.

17. A method according to claim 9, wherein said second gene-specific probe comprises the sequence set forth in SEQ ID NO:8.

18. A method of diagnosing an infection in individual as being HSV-1 or HSV-2, said method comprising, detecting a first target sequence specific for HSV-1 when HSV-1 nucleic acids are present in a biological sample from said individual, and detecting a second target sequence specific for HSV-2 when HSV-2 nucleic acids are present in said sample, wherein said first target sequence and said second target sequence are from different genes, and wherein detection of said first target sequence is indicative of an HSV-1 infection and detection of said second target sequence is indicative of an HSV-2 infection.

19. A method according to claim 18, wherein said first target sequence is from the HSV-1 glycoprotein B gene.

20. A method according to claim 18, wherein said second target sequence is from the HSV-2 UL-8 gene.

21. A method according to claim 18, wherein said first target sequence is from the HSV-1 glycoprotein B gene and said second target sequence is from the HSV-2 UL-8 gene.

22. A method according to claim 18, wherein said detecting further comprises, amplifying said first and second target sequences.

23. A method according to claim 22, wherein said amplifying is accomplished with the polymerase chain reaction.

24. A method according to claim 18, wherein said detecting of said first and second target sequences occurs in the same reaction vessel.

25. A method according to claim 18, wherein said detecting of said first and second target sequences occurs in separate reaction vessels.

26. A method according to claim 18, wherein said detecting further comprises,
amplifying a region of the HSV-1 glycoprotein B (gB) gene with a first pair of type-specific primers when HSV-1 nucleic acids are present in said sample, amplifying a region of the HSV-2 UL-8 gene with a second pair of type-specific primers when HSV-2 nucleic acids are present in said sample, and
detecting said amplified regions with a first gene-specific probe and a second gene-specific probe.

27. A method according to claim 26, wherein said probes are labeled.

28. A method according to claim 27, wherein said probes are distinguishably labeled.

29. A method according to claim 26, wherein said first pair of type-specific primers comprises a forward primer comprising the sequence set forth in SEQ ID NO:3 and a reverse primer.

30. A method according to claim 26, wherein said first pair of type-specific primers comprises a forward primer and a reverse primer comprising the sequence set forth in SEQ ID NO:4.

31. A method according to claim 26, wherein said second pair of type-specific primers comprises a forward primer comprising the sequence set forth in SEQ ID NO:6 and a reverse primer.

32. A method according to claim 26, wherein said second pair of type-specific primers comprises a forward primer and a reverse primer comprising the sequence set forth in SEQ ID NO:7.

33. A method according to claim 26, wherein said first gene-specific probe comprises the sequence set forth in SEQ ID NO:5.

34. A method according to claim 26, wherein said second gene-specific probe comprises the sequence set forth in SEQ ID NO:8.
